(19) United States
(12) Patent Application Publication
(10) Pub. No.: US 2019/0174790 A1
(43) Pub. Date: Jun. 13, 2019

(54) ESPRESSO BEVERAGE FORMULATION COMPRISING COLLAGEN PEPTIDES

(71) Applicant: Gil NOFAR, Haifa (IL)

(72) Inventor: Gil NOFAR, Haifa (IL)

(21) Appl. No.: 15/742,977

(22) PCT Filed: Jul. 7, 2016
(86) PCT No.: PCT/IL2016/050734

§ 371 Date: Jan. 9, 2018

Related U.S. Application Data
(60) Provisional application No. 62/190,286, filed on Jul. 9, 2015.
(60) Provisional application No. 62/244,784, filed on Oct. 22, 2015.

(51) Int. Cl. A23F 5/14; B65D 85/804; B65B 29/02; C07K 14/78

(52) U.S. Cl. CPC A23F 5/14; C07K 14/78; B65B 29/022; B65D 85/8043

(57) ABSTRACT

The present invention provides an espresso cartridge for the preparation of an espresso beverage containing collagen peptides, the espresso cartridge contains a portion of a substance for the preparation of an espresso beverage and a portion of collagen powder. The present invention further provides a method for preparing an espresso cartridge for the preparation of an espresso beverage containing collagen peptides, the method comprises inserting into an espresso cartridge a portion of a substance for the preparation of an espresso beverage and a portion of collagen powder.

### CROSS-REFERENCE TO RELATED APPLICATIONS

The present application claims priority to U.S. provisional patent application No. 62/190,286, filed Jul. 9, 2015 and U.S. provisional patent application No. 62/244,784, filed Oct. 22, 2015.

### FIELD OF THE INVENTION

The present invention relates to formulations comprising collagen peptides. More particularly, the present invention relates to formulations comprising collagen peptides for the preparation of espresso beverages containing collagen peptides.

### BACKGROUND OF THE INVENTION

Collagen is a structural protein found in the extracellular space in various connective tissues in animal bodies. A formulation comprising hydrolyzed collagen peptides, for example in the form of powder, is the bioavailable form of collagen. In general, the process of preparing a hydrolyzed collagen peptides powder involves cutting of bones, skin and connective tissue of animals that are rich with collagen, followed by treatment with acid, enzymatic hydrolysis, high pressure spraying, drying, blending and sieving. Two types of hydrolyzed collagen peptides powder are available: glomerate powder comprising higher sized particles, and spray dried powder comprising finer particles. Commercially available hydrolyzed collagen peptides powder may comprise collagen peptides in different molecular weights, for example 2000, 3000 or 5000 Da.

Clinical trials have shown that oral administration of hydrolyzed collagen peptides can be effective in improving skin moisture, elasticity, texture and clarity; reducing wrinkles, skin roughness and Ultra-Violet (UV) spots; increasing hair thickness as well as alleviating osteoarthritis and improving bone density.

Orally administered supplements containing hydrolyzed collagen peptides are commercially available in the form of pill or powder. Hydrolyzed collagen peptides pills are directly ingested by a user. Hydrolyzed collagen peptides powder is mixed with a beverage serving, for example tea, coffee, milk, juice and the like, and ingested by a user together with the beverage. Another way of consuming the hydrolyzed collagen peptides powder is mixing the powder with the ingredients of a foodstuff, for example chocolate bars, cakes and the like, and ingesting the collagen peptides together with the foodstuff.

A randomized double-blind placebo-controlled clinical trial of ingestion of two types of collagen hydrolysates was conducted by Inoue et al (2016). During the study, participants orally ingested 5 gr samples of collagen hydrolysate, or placebo, in the form of powder, in hot milk, coffee, or any other beverages, once a day after dinner for 8 weeks. The collagen hydrolysates, which are commercially available under the Wellnex brand, were derived from fish gelatin, and were manufactured by Nitta Gelatin Inc. (Osaka, Japan). Participant ingesting the collagen powder showed improvement in skin conditions, such as skin moisture, elasticity, wrinkles, and roughness, during the study period, compared to participant ingesting placebo powder (Inoue, N., Sugihara, F. and Wang, X, Ingestion of bioactive collagen hydrolysates enhance facial skin moisture and elasticity and reduce facial ageing signs in a randomized double-blind placebo-controlled clinical study, Journal of the Science of Food and Agriculture, doi: 10.1002/jsfa.7606, published 8 Jan. 2016). These results demonstrate that ingestion of a daily dosage of 5 gr hydrolyzed collagen peptides powder is effective in terms of skin conditions.

In another double-blind, placebo controlled trial, conducted by Proksch et al (2013), participants orally ingested collagen hydrolysates or placebo during a period of 8 weeks. The collagen hydrolysate composed of different specific collagen peptides derived from a special hydrolysis of porcine type I collagen. The product was manufactured by GELITA AG (Eberbach, Germany), commercially available under the name VERISOL®. The average molecular weight of the collagen peptides in the collagen hydrolysate was 2000 Da. A daily dose of either 2.5 or 5.0 gr of collagen hydrolysate, or 2.5 gr placebo, in the form of powder was ingested by dissolving the powder in water or any other cold liquid. During the trial period, skin elasticity in both collagen hydrolysate dosage groups showed a significant improvement in comparison to placebo, and after 4 weeks of follow-up treatment, a statistically significantly higher skin elasticity level was determined in elderly women (Proksch, E., Segger, D., Degwert, J., Schunck, M., Zangue, V and Oesser, S., Oral supplementation of specific collagen peptides has beneficial effects on human skin physiology: A double-blind, placebo-controlled study, Skin Pharmacology and Physiology, 27, 47-55. Published 14 Aug. 2013). These results indicate that a daily dosage of 2.5 gr of hydrolyzed collagen peptides powder comprising collagen peptides having a molecular weight of 2000 Da is still effective in terms of skin conditions, specifically skin elasticity.

Other experiments have demonstrated that, in general, a lower molecular weight of hydrolyzed collagen peptides gives rise to a lower daily effective dosage of the hydrolyzed collagen peptides, while a higher molecular weight of hydrolyzed collagen peptides gives rise to a higher daily dosage of the hydrolyzed collagen peptides that has to be ingested in order to affect skin conditions and other targets of collagen peptides. For example, a minimal daily ingestion of 5 gr of hydrolyzed collagen peptides powder comprising hydrolyzed collagen peptides having a molecular weight of 5000 Da is effective, while a minimal daily ingestion of only 3 gr hydrolyzed collagen peptides powder comprising hydrolyzed collagen peptides having a molecular weight of 3000 Da is effective.

Based on these studies, suppliers of hydrolyzed collagen peptides powder recommend daily ingestion of at least substantially 3 gr hydrolyzed collagen peptides powder comprising peptides with a molecular weight of 3000 Da, and more particularly in the range of substantially 3-10 gr daily, in order to affect the skin and improve joints health. In additions, according to the results obtained by Proksch et al (2013), a daily dosage of 2.5 gr collagen peptides having a molecular weight of 2000 Da is sufficient from improving skin conditions. Such high amounts of hydrolyzed collagen peptides powder that have to be ingested on a daily basis may discourage potential consumers from ingesting hydrolyzed collagen peptides powder. Another factor that may adversely affect the level of ingestion of hydrolyzed collagen peptides powder is the texture and taste of hydrolyzed collagen peptides powder that is unpleasant to the palate of some consumers.

Thus, the barrier of supplying a daily effective dosage of collagen peptides to a user while eliminating the discomfort of ingesting high amounts of hydrolyzed collagen peptides powder, having an unpleasant texture and taste, on a daily basis, has still to be overcome.

Therefore, there is a need for a formulation of hydrolyzed collagen peptides powder that allows ingestion of large amounts of hydrolyzed collagen peptides powder on a daily basis without causing a filling of burden to the consumer in one hand, and overcome the barrier of unpleasant texture on the other hand.

### SUMMARY OF THE INVENTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

According to one aspect of the present invention, there is provided an espresso cartridge for the preparation of an espresso beverage containing collagen peptides, the espresso cartridge contains a portion of a substance for the preparation of an espresso beverage and a portion of collagen powder.

According to another aspect of the present invention, there is provided a method for preparing an espresso cartridge for the preparation of an espresso beverage containing collagen peptides, the method comprises inserting into an espresso cartridge a portion of a substance for the preparation of an espresso beverage and a portion of collagen powder.

According to one embodiment, the portion of the substance for the preparation of an espresso beverage and the portion of collagen powder are mixed.

According to another embodiment, the portion of substance for the preparation of an espresso beverage and the portion of collagen powder are separated in a plurality of layers.

According to yet another embodiment, at least one layer comprises a portion of a substance for the preparation of an espresso beverage, and at least one layer comprises a portion of collagen powder.

According to still another embodiment, at least one layer comprises a mixture of a portion of substance for the preparation of an espresso beverage and a portion of collagen powder, and at least one layer comprises a portion of a substance for the preparation of an espresso beverage.

According to a further embodiment, at least one layer comprises a mixture of a portion of a substance for the preparation of an espresso beverage and a portion of collagen powder, and at least one layer comprises a portion of collagen powder.

According to yet a further embodiment, at least one layer comprises a mixture of a portion of a substance for the preparation of an espresso beverage and a portion of collagen powder, at least one layer comprises a portion of a substance for the preparation of an espresso beverage, and at least one layer comprises a portion of collagen powder.

According to an additional embodiment, a layer comprising a portion of a substance for the preparation of an espresso beverage is placed adjacent to an exit side of the espresso cartridge, and a layer comprising a portion of collagen powder is placed adjacent to an entry side of the espresso cartridge.

According to yet an additional embodiment, a layer comprising a portion of collagen powder is placed adjacent to an exit side of the espresso cartridge, and a layer comprising a portion of a substance for the preparation of an espresso beverage is placed adjacent to an entry side of the espresso cartridge.

According to one embodiment, an amount of collagen powder in the espresso cartridge is at least substantially 0.2 gr.

According to another embodiment, an amount of collagen powder in the espresso cartridge is at least substantially 1.5 gr.

According to one embodiment, an amount of collagen powder in the espresso cartridge is in the range of substantially 3-100% of a total amount of substance in the espresso cartridge.

According to another embodiment, an amount of collagen powder in the espresso cartridge is in the range of substantially 3-83% of a total amount of substance in the espresso cartridge.

According to yet another embodiment, an amount of collagen powder in the espresso cartridge is in the range of substantially 3-50% of a total amount of substance in the espresso cartridge.

According to still another embodiment, an amount of collagen powder in the espresso cartridge is in the range of substantially 3-42% of a total amount of substance in the espresso cartridge.

According to a further embodiment, an amount of collagen powder in the espresso cartridge is in the range of substantially 12-33% of a total amount of substance in the espresso cartridge.

According to yet a further embodiment, an amount of collagen powder in the espresso cartridge is in the range of substantially 16-33% of a total amount of substance in the espresso cartridge.

According to still a further embodiment, an amount of collagen powder in the espresso cartridge is substantially 25% of a total amount of powder in the espresso cartridge.

According to an additional embodiment, the espresso cartridge further comprises at least one additive.

According to yet an additional embodiment, the at least one additive is selected from the group consisting of nutritional supplement, flavor enhancer, health enhancer, and any combination thereof.

According to still an additional embodiment, the at least one additive is selected from the group consisting of lemon lime, Muscat nut, cinnamon, coffee berry, coffee berry extract, hyaluronic acid, glycyrrhizinate (licorice extract), derivative products of glycyrrhizinate, and any combination thereof.

### DESCRIPTION OF THE PREFERRED EMBODIMENTS

Before explaining at least one embodiment in detail, it is to be understood that the invention is not limited in its application to the details of construction and the arrangement of the components set forth in the following description. The invention is capable of other embodiments or of being practiced or carried out in various ways. Also, it is to be understood that the phraseology and terminology employed herein is for the purpose of description and should not be regarded as limiting.

The term “substance for the preparation of an espresso beverage”, as disclosed herein, refers to any substance known in the art that is suitable for the preparation of an espresso beverage, as disclosed hereinafter. A substance for the preparation of an espresso beverage may be in a form of a powder. Examples of a powder for the preparation of an espresso beverage, according to the present invention, include but not limited to, ground coffee beans powder, ground cocoa beans powder, and the like. In addition, a substance for the preparation of an espresso beverage may be in the form of particles, for example, but not limited to, intact or chopped leaves—like tea leaves, mint leaves, and the like.

The term “espresso beverage”, as disclosed herein, according to one embodiment refers to a beverage brewed by applying nearly boiling water through a substance for the preparation of an espresso beverage, and according to another embodiment refers to a beverage brewed by forcing a small amount of nearly boiling water under pressure through a substance for the preparation of an espresso beverage. The term “espresso beverage” further refers to an espresso beverage made by any method and device known in the art, including, but not limited to, filter coffee machines, espresso machines, heavy duty espresso machines, espresso machine that also grind coffee beans, household or home espresso machines, espresso machines using cartridges containing a substance for the preparation of an espresso beverage, espresso machines using espresso pods containing a substance for the preparation of an espresso beverage, espresso machines using espresso pads containing a substance for the preparation of an espresso beverage, and the like. The term “espresso beverage” further refers to any type of beverage made on the basis of an espresso beverage described above, for example but not limited to, caffe latte, cappuccino, caffe macchiato, caffe mocha, flat white, caffe Americano, and the like.

The term “cartridge” or “espresso cartridge”, as disclosed herein, refers to any container known in the art, that may contain a substance for the preparation of an espresso beverage, that is suitable for the preparation of an espresso beverage by any method and any device known in the art for the preparation of an espresso beverage. Examples of a cartridge include, but not limited to: a container made of a rigid material for placement of a substance for the preparation of an espresso beverage, for example a container made of metal, configured for usage with espresso machines like heavy duty espresso machines; a disposable container (usually termed “espresso cartridge” or “espresso capsule”) containing a substance for the preparation of an espresso beverage; an espresso pod, and espresso pad, and the like. Examples of commercially available disposable espresso cartridges that are under the scope of the present invention, include, but not limited to, Lavazza Espresso Capsules, K-Cup® Pods, K-Cup® Reusable Coffee Filters, IperEspresso Capsules (Illy), Nespresso Capsules, and NESCAFE® Dolce Gusto® Capsules.

The term “collagen powder”, as disclosed herein, refers to any hydrolyzed collagen peptides powder known in the art, including inter alia collagen hydrolysate of the type gelatin 0 Bloom.

The term “coffee powder”, as disclosed herein, refers to any ground coffee beans powder known in the art, suitable for the preparation of an espresso beverage.

As described previously, the recommended effective daily dosage of collagen peptides having a molecular weight of 3000 Da is at least substantially 3 gr, and the effective daily dosage of collagen peptides having a molecular weight of 2000 Da is at least substantially 2.5 gr. For the sake of simplicity only, and in order to be on the safe side in terms of effectiveness on consumers, the recommended daily dosage of collagen peptides refers herein to an amount of at least 3 gr collagen peptides.

According to one aspect, the present invention provides a formulation for the preparation of an espresso beverage containing collagen peptides, the formulation comprising a substance for the preparation of an espresso beverage and a collagen powder. Furthermore, the present invention provides an espresso cartridge containing a formulation for the preparation of an espresso beverage containing collagen peptides, the formulation comprising a substance for the preparation of an espresso beverage and a collagen powder, and a method for the preparation thereof. Furthermore, the formulation of the present invention comprises a mixture of a substance for the preparation of an espresso beverage and a collagen powder. In addition, the present invention provides an espresso cartridge containing a formulation for the preparation of an espresso beverage containing collagen peptides, wherein the cartridge comprises a layer of a powder for the preparation of an espresso beverage and a layer of collagen powder; and a method for the preparation thereof. Furthermore, the present invention provides an espresso cartridge containing a formulation of the preparation of an espresso beverage containing collagen peptides, wherein the cartridge comprises a layer of a powder for the preparation of an espresso beverage and a layer of a mixture of a powder for the preparation of an espresso beverage and a collagen powder; and a method for the preparation thereof.

Commercially available espresso cartridges may be in various shapes and sizes. Nevertheless, all espresso cartridges share several common structural and functional features. An espresso cartridge is a container configured to contain a substance for the preparation of an espresso beverage. One side of the espresso cartridge, termed hereinafter “entry side”, allows entry of water, for example hot water, into the cartridge. Then, the water passes through the substance for the preparation of an espresso beverage contained in the cartridge, and water soluble components of the substance for the preparation of an espresso beverage dissolve in the water. A beverage comprising a solution of the water soluble components of the substance for the preparation of an espresso beverage exit the cartridge through a side of the espresso cartridge, termed hereinafter “exit side”, into a beverage container, for example, but not limited to, a cup, a jug, and the like.

According to some embodiments, the present invention provides a formulation for the preparation of an espresso beverage containing collagen peptides. According to other embodiments, the present invention provides an espresso cartridge for the preparation of an espresso beverage containing collagen peptides. According to one embodiment, an espresso beverage serving prepared with the formulation or the cartridge of the present invention provides at least substantially 0.2 gr collagen peptides, which is substantially 6% of the recommended minimal effective daily dosage of 3 gr collagen peptides.

Thus, according to one embodiment, the present invention provides a formulation for the preparation of an espresso beverage serving comprising at least substantially 0.2 gr collagen powder. According to another embodiment, the present invention provides an espresso cartridge for the preparation of an espresso beverage serving, wherein the cartridge contains a formulation for the preparation of an espresso beverage serving comprising at least substantially 0.2 gr collagen powder. According to yet another embodiment, the present invention provides an espresso cartridge for the preparation of an espresso beverage serving, wherein the cartridge contains a formulation for the preparation of an espresso beverage serving comprising at least substantially 1.5 gr collagen powder, which is at least half of the recommended effective daily dosage of 3 gr collagen peptides. Ingestion of at least two servings of espresso beverage containing collagen peptides, prepared with the aforementioned formulation and cartridge containing at least substantially 1.5 gr collagen powder, provides the recommended effective daily dosage of collagen.

Hereinafter, examples of experiments conducted during the development of the present invention are described. During the experiments, coffee powder as a substance for the preparation of an espresso beverage, a standard disposable Nespresso compatible cartridge (capable of containing up to 13.5 ml water), an espresso device suitable for the usage of standard disposable Nespresso compatible cartridges, and various types of collagen powder, were used. It should be noted that these examples should not be regarded as limiting the scope of the present invention, but rather regarded as a means for demonstrating the efficacy of the present invention.

For the sake of simplicity, any reference to a “Nespresso cartridge” in the description of the experiments is to a standard disposable Nespresso compatible cartridge capable of containing up to 13.5 ml water.

A Nespresso cartridge normally contains substantially 5-6 gr coffee powder for the preparation of an espresso coffee beverage serving. Preliminary experiments were conducted with a mixture of substantially 4.5 gr coffee powder and substantially 1.5 gr collagen power per Nespresso cartridge, which provides half of the minimal recommended daily dosage of collagen peptides. Except only a few cases, no exit, or very poor exit, of an espresso beverage from the Nespresso cartridge was observed. On the other hand, Nespresso cartridges containing 6 gr collagen powder allowed proper exit of an espresso beverage from the Nespresso cartridge. In addition, as well known in the art, a Nespresso cartridge containing substantially 6 gr of coffee powder allows proper exit of an espresso beverage from the Nespresso cartridge. Thus, these results demonstrate that in a mixture of coffee powder and collagen powder there is a tendency of clogging the passage of water through the Nespresso cartridge—a phenomenon preventing a preparation of an espresso beverage containing collagen peptides.

Surprisingly, it was discovered that placing the coffee powder and the collagen powder in separate layers in the Nespresso cartridge dramatically improves the exit of an espresso beverage from the Nespresso cartridge.

Since one aim of the present invention is to provide an espresso beverage that provides at least substantially 6% of the recommended effective daily dosage of collagen, one has to show that the espresso beverage contains collagen peptides. Table 1 shows the amount of protein in a serving of 100 ml espresso beverage prepared with Nespresso cartridges manually filled with collagen powder having a molecular weight of either 2000 Da or 3000 Da and coffee powder, differing in the relative orientation of the layers of coffee powder and collagen powder in the Nespresso cartridge.

A 100 ml espresso beverage serving was prepared with each of the various types of Nespresso cartridges, with the number of repetitions (n) indicated in Table 1. Then the total amount of protein in each of the espresso beverage servings was determined, using the AOAC Official Method 981.10 for the measurement of crude protein in meat. The amounts of protein shown in Table 1 are average amounts±standard deviation.

TABLE 1
Amount of protein in 100 ml espresso beverage
servings prepared with Nespresso cartridges containing
coffee powder and collagen powder.

| | | | Protein in 100 ml Espresso |
|---|---|---|---|
| Cartridge | Position * | Content of Cartridge | Beverage Serving (gr) |
| A | Entry | 4.5 gr coffee ** | 0.265 ± 0.009 |
| | Exit | | n = 3 |
| B | Entry | 1.5 gr collagen 2000 | 1.625 ± 0.071 |
| | Exit | 4.5 gr coffee | n = 5 |
| C | Entry | 4.5 gr coffee | 1.184 ± 0.128 |
| | Exit | 1.5 gr collagen 2000 | n = 3 |
| D | Entry | 1.5 gr collagen 3000 | 1.693 ± 0.206 |
| | Exit | 4.5 gr coffee | n = 5 |
| E | Entry | 4.5 gr coffee | 0.819 ± 0.074 |
| | Exit | 1.5 gr collagen 3000 | n = 3 |

* The “Position” column designates the side of the Nespresso cartridge near which the layer of coffee powder or collagen powder is placed. “Entry” means that the layer is adjacent to the entry side of the Nespresso cartridge. “Exit” means that the layer is adjacent to the exit side of the Nespresso cartridge.
** Cartridge A contained substantially 6 gr coffee powder. However, the amount of protein in the espresso beverage prepared with cartridge A was normalized to a Nespresso cartridge containing substantially 4.5 gr coffee powder, in order to be comparable to cartridges B, C, D and E that contain substantially 4.5 gr coffee powder.

As can be seen in Table 1, an espresso beverage serving prepared with substantially 4.5 gr coffee powder (cartridge A), contained a basic amount of protein—considered as background level of protein in the beverage. On the other hand, the espresso beverage servings prepared with cartridges B-E, containing a layer of substantially 4.5 gr coffee powder and a layer of substantially 1.5 gr collagen powder, contained much higher amounts of protein. These results indicate that an espresso beverage prepared with a Nespresso cartridge containing collagen powder contains collagen peptides that may be ingested by a user while ingesting the espresso beverage.

In this regard, it was observed that there is a correlation between the quality of flow of an espresso beverage from a Nespresso cartridge and the amount of collagen peptides in the espresso beverage. The better the quality of flow is—the higher is the amount of collagen peptides in the espresso beverage.

The results in Table 1 further show that the relative positioning of the layers in the cartridge affects the amount of protein transferred to the espresso beverage serving. When the layer of coffee powder is positioned adjacent to the exit side of the cartridge, and the layer of collagen powder is positioned adjacent to the entry side of the cartridge (cartridges B and D), the amount of protein in the espresso beverage is substantially higher in comparison to espresso beverage servings prepared with cartridges with an opposite orientation of the layers (cartridges C and E). These results indicate that positioning of a layer of coffee powder adjacent to the exit side of the cartridge and a layer of collagen powder adjacent to the entry side of the cartridge gives rise to a higher yield of collagen peptides in the espresso beverage serving in comparison to an opposite positioning of the layers in the cartridge.

Furthermore, cartridges containing a layer of coffee powder and a layer of a mixture of coffee powder and collagen powder allowed proper exit of an espresso beverage from the cartridge. The layer of coffee powder may be positioned either adjacent to the exit side or the entry side of the cartridge.

The experiments shown in Table 1 were conducted with substantially 1.5 gr collagen powder and substantially 4.5 gr coffee powder per Nespresso cartridge, giving rise to a total amount of substantially 6 gr powder for the preparation of an espresso beverage per Nespresso cartridge. In all the experiments shown in Table 1, there was proper exit of an espresso beverage from the Nespresso cartridges. Similar experiments with other amounts of collagen powder per Nespresso cartridge, while keeping the total amount of powder per Nespresso cartridge at substantially 6 gr were conducted as well, and the exit of an espresso beverage from the Nespresso cartridge was examined. Lower amounts of collagen powder per cartridge—down to substantially 0.2 gr collagen powder per Nespresso cartridge (and substantially 5.8 gr coffee powder per Nespresso cartridge), and higher amounts of collagen powder per Nespresso cartridge—up to substantially 6 gr collagen powder per Nespresso cartridge (without coffee powder), gave rise to proper exit of an espresso beverage from the Nespresso cartridge. Nevertheless, in general lower levels of collagen powder per cartridge give rise to better flow of beverage from the cartridge.

Based on these results, the present invention provides the following ranges of weights of collagen powder per cartridge having a powder for the preparation of an espresso beverage, for example coffee powder, cocoa powder and the like, and collagen powder, when the total weight of powder per cartridge is substantially 6 gr: substantially 0.2-6 gr, 0.2-5 gr, 0.2-3 gr, 0.2-2.5 gr, 0.75-2 gr, 1-2 gr and 1.5 gr collagen powder per cartridge.

Since the scope of the invention includes all types of espresso cartridges, which may enable insertion of various total amounts of powder into the cartridge, the present invention provides the following ranges of percentage of collagen powder from total amount of powder in a cartridge: substantially 3-100%, 3-83%, 3-50%, 3-42%, 12-33%, 16-33% and 25% collagen powder of total amount of powder per cartridge.

It should be noted that during experiments conducted by the inventor, it has been found that the amount of collagen powder that may be inserted into a cartridge depends on the size of the particles of the collagen powder. In general, higher sizes of collagen powder particles allow the insertion of lower amounts of collagen powder into a cartridge and vice versa.

Using a cartridge containing for example a layer of substantially 2.5 gr collagen powder and a layer of substantially 3.5 gr coffee powder is beneficial, especially for users that prefer to consume an espresso beverage with a low content of coffee, while providing the minimal recommended effective daily dosage of collagen peptides having a molecular weight of 2000 Da (Proksch et al, 2013). Using a cartridge containing a layer of substantially 1.5 gr collagen powder and a layer of substantially 4.5 gr coffee beans powder is optimal in terms of the amount of collagen consumed by the user—approximately half of the recommended daily effective dosage—thus a daily ingestion of two such espresso beverage servings provides a daily dosage of collagen peptides of 3 substantially gr, which is a bit higher than the recommended minimal effective daily dosage of collagen peptides having a molecular weight of 2000 Da. However, an amount of coffee powder lower than substantially 5 gr per cartridge gives rise to a coffee espresso beverage having an insufficient level of taste and aroma for an average coffee consumer. One may increase the amount of coffee powder to substantially 5 gr or higher per cartridge, however it comes at the expanse of the amount of collagen powder per cartridge.

In this regard, the inventor has surprisingly discovered that further grinding of the collagen powder enabled, in a Nespresso cartridge, packaging of a layer of substantially 1.5 gr ground collagen powder and a layer of substantially 4.8-5 gr coffee powder—giving rise to a cartridge having optimal amounts of collagen peptides and coffee. In this example, a collagen powder (molecular weight 2000 Da) was used. Examination of the ground collagen powder under a magnifying device, like a microscope or a magnifying glass, revealed that unlike non-ground collagen powder, the ground collagen powder comprises particles having irregular shapes. Furthermore, as can be seen in Table 2, the size of the ground collagen powder particles is lower than the size of the non-ground collagen powder particles. The size of the ground and non-ground collagen powder particles was determined with a Mastersizer 3000 particle sizing instrument (Malvern Instruments Limited, Worcestershire, UK). This instrument measures the diameter of a population of particles, and determines the distribution of the diameters of the particles in the population, which is a normal distribution. The diameter of the particles in the following tables is expressed as the 10^{th} percentile diameter, 50^{th} percentile (median) diameter and 90^{th} percentile diameter. In some cases, only the median diameter is given.

TABLE 2
Size distribution of commercially available collagen powder
particles and ground collagen powder particles.

| | Particles Diameter (μm) | | |
|---|---|---|---|
| Collagen 2000 | 10^{th} Percentile | 50^{th} Percentile | 90^{th} Percentile |
| Original (Not ground) | 43.4 | 131 | 304 |
| Ground (Batch #1) | 31.4 | 104 | 232 |
| Ground (Batch #2) | 30.0 | 94.9 | 204 |

Further experiments were conducted—examining the quality of flow of exit of an espresso beverage from Nespresso cartridges containing various types of coffee powder differing in particle diameter, and various types of collagen powder differing in the molecular weight of the collagen peptides, the manufacturer and the particle diameter. The results are summarized in Table 3. All the Nespresso cartridges were manually filled with a layer of substantially 1.5 gr collagen powder adjacent to the entry side of the cartridge, and a layer of coffee powder adjacent to the exit side of the cartridge. In most of the cases the amount of coffee powder that could be inserted into the Nespresso cartridge was substantially 4.5 gr, as shown in Table 3. However, in some cases, that will be discussed later, other amounts—lower or higher, of coffee powder could be inserted into the Nespresso cartridge, in correlation with the particles' diameter, as shown in Table 3.

The quality of flow of exit of an espresso beverage from a cartridge was evaluated by observing the width and flow rate of a stream of the espresso beverage exiting the cartridge. A minus (−) sign designates no flow of an espresso beverage from the cartridge. Up to four plus (+) signs designate flow of an espresso beverage from the cartridge. A higher number of plus (+) signs designates a higher quality of the stream of espresso beverage, in term of width of stream and flow rate.

Table 3 also shows three types of collagen powder and two types of coffee powder. “Original” designates a commercial collagen or coffee powder used without any treatment; “Ground X1” designates a commercial collagen or coffee powder ground once with an industrial scale grinding machine; and “Ground X2” designates a commercial collagen powder ground twice with an industrial scale grinding machine. It should be noted that there is some variation in the size of the particles following repetitions of a grinding process with similar parameters, for example speed and duration.

TABLE 3
Flow of an espresso beverage from Nespresso cartridges containing a layer of coffee
powder - containing particles in different sizes, adjacent to an exit side of the cartridge; and a
layer of collagen powder - from different manufacturers and containing peptides in different
molecular weight and particles in different sizes, adjacent to an entry cover of the cartridge.

| Collagen Powder | | | | Coffee Powder | | |
|---|---|---|---|---|---|---|
| 1.5 gr/Cartridge | | | | Ground X1 | Original | |
| | | | Size (μm) | (10^{th}, 50^{th}, 90^{th}) | (10^{th}, 50^{th}, 90^{th}) | gr/ |
| Source | MW | Type | (10^{th}, 50^{th}, 90^{th}) | 43, 386, 728 | 84, 464, 1220 | Cartridge |
| Nitta, | 5000 | Original | 112, 279, 562 | + | − | 3.8 |
| China #1 | | Ground X1 | 102, 230, 394 | + | − | 3.9 |
| Nitta, | 5000 | Original | 34, 91, 190 | +++ | − | 4.5 |
| China #2 | | Ground X1 | 30, 86, 184 | ++++ | − | 4.5 |
| Rousselot, | 5000 | Original | 50, 119, 233 | + | − | 4.5 |
| France | | Ground X1 | 42, 106, 205 | ++ | − | 4.5 |
| Nitta, | 3000 | Original | 52, 138, 323 | + | − | 4.5 |
| India | | Ground X1 | 41, 111, 241 | ++ | − | 4.5 |
| | | Ground X2 | 36, 99, 213 | ++++ | − | 4.5 |
| | | Ground X2 | 36, 99, 213 | ++ | − | 4.8 |
| Nitta, | 3000 | Original | 53, 137, 285 | ++ | − | 4.5 |
| China | | Ground X1 | 38, 108, 216 | ++ | − | 4.5 |
| | | Ground X2 | 35, 99, 192 | ++++ | − | 4.5 |
| | | Ground X2 | 35, 99, 192 | +++ | − | 4.8 |
| | | Ground X2 | 35, 99, 192 | ++ | − | 5 |
| Rousselot, | 2000 | Original | 43, 131, 304 | ++ | − | 4.5 |
| France | | Ground X1 | 31, 104, 233 | +++ | − | 4.5 |
| | | Ground X2 | 30, 96, 202 | +++ | − | 4.8 |
| | | Ground X2 | 30, 96, 202 | + | − | 5 |

Effect of Coffee Powder Particle Size on the Quality of Flow of an Espresso Beverage Containing Collagen Peptides from a Cartridge

Espresso beverages containing collagen peptides were prepared with Nespresso cartridges containing various types of collagen powder, and either a coffee powder containing large particles (10^{th}, 50^{th} and 90^{th} percentile sizes of 84, 464 and 1220 μm, respectively) or smaller particles (10^{th}, 50^{th} and 90^{th} percentile sizes of 43, 386 and 728 μm, respectively). According to the results shown in Table 3, all the Nespresso cartridges containing large particles of coffee powder did not allow exit of an espresso beverage from them. On the other hand, Nespresso cartridges containing smaller particles of coffee powder allowed exit of an espresso beverage from them in the presence of all the types of collagen powder that were tested.

It should be noted that these results should be considered as exemplary only, and other results may be obtained under other conditions, for example when using other types of the substance for the preparation of an espresso beverage, like coffee powder, other types of collagen powder, other sizes of particles of the substance for the preparation of an espresso beverage and collagen powder, and other types of espresso cartridges. Furthermore, the pressure in which the substance for the preparation of an espresso beverage and the collagen powder are filled-in in the cartridge may also influence the exit of espresso beverage from the cartridge.

### Amount of Coffee Powder Per Cartridge

In order to obtain an espresso beverage containing collagen peptides it is recommended to use a powder for the preparation of an espresso beverage comprising smaller particles, rather than larger particles—For example, using a coffee powder comprising particles having 10^{th}, 50^{th} and 90^{th} percentile sizes of 43, 386 and 728 μm, respectively, rather than using a coffee powder comprising larger particles, for example particles having 10^{th}, 50^{th} and 90^{th} percentile sizes of 84, 464 and 1220 μm, respectively.

It should be noted that these particle sizes of coffee powder should not be considered as limiting the scope of the present invention, but rather be considered as examples only to demonstrate the importance of the size of the particles of a powder for the preparation of an espresso beverage when used for the preparation of an espresso beverage containing collagen peptides.

Effect of Collagen Powder Particle Size on the Quality of Flow of an Espresso Beverage Containing Collagen Peptides from a Cartridge

The results shown in Table 3 demonstrate a general correlation between the size of the collagen powder particles and the quality of flow of an espresso beverage from the cartridge. A higher particles size gives rise to a poor flow of the espresso beverage from the cartridge, and a smaller particle size gives rises to a better flow of the espresso beverage from the cartridge.

Therefore, in order to obtain a good quality flow of espresso beverage containing collagen peptides it is recommended to use a collagen powder having small particles, for example but not limited to, particles having a median particle size of substantially 100 μm. Nevertheless, it should be noted that another size of particle may apply to combinations of other types of cartridge, coffee powder and collagen powder.

It should be noted that the general correlation between the size of the collagen powder particles and the quality of flow of an espresso beverage from the cartridge, as described above, should not be considered as limiting the scope of the invention, and that in some conditions such a correlation may not apply. Furthermore, the particle sizes of collagen powder mentioned herein should not be considered as limiting the scope of the present invention, but rather be considered as examples only to demonstrate the importance of the size of the particles of a powder for the preparation of an espresso beverage when used for the preparation of an espresso beverage containing collagen peptides.

As discussed previously, a Nespresso cartridge is normally capable of containing totally substantially 6 gr coffee powder. Substituting substantially 1.5 gr coffee powder per Nespresso cartridge with a similar amount of collagen powder results in an espresso beverage that may not be accepted by users due to a low level of coffee taste and aroma. Therefore, increasing the amount of coffee powder per cartridge, in the presence of the same amount of collagen powder is advantageous. In this regard, it was found by the inventor that grinding collagen powder twice allows increasing the amount of coffee powder that may be inserted into a Nespresso cartridge, in the presence of substantially 1.5 gr collagen powder, when the molecular weight of the collagen peptides is 3000 Da or 2000 Da.

Table 3 further shows the quality of flow of the espresso beverage exiting the tested cartridges. The results show a correlation between the amount of coffee powder per cartridge and the quality of flow of an espresso beverage made with substantially 1.5 gr collagen powder per Nespresso cartridge. In the presence of up to substantially 4.8 gr coffee powder per Nespresso cartridge the flow quality of the espresso beverage from the Nespresso cartridge was better than in the presence of substantially 5 gr coffee powder per Nespresso cartridge.

This experiment and others indicate that the flow of an espresso beverage from a cartridge containing coffee powder and collagen powder depends on the total amount of powder in the cartridge. In general, a lower total amount of powder in the cartridge allows proper flow of an espresso beverage from the cartridge, and a higher total amount of powder in the cartridge makes the flow of an espresso beverage from the cartridge more difficult. The reason for this may be that in order to insert a higher amount of powder in the cartridge the powder should be more compressed, thus increasing the chance of clogging the cartridge and preventing proper flow of an espresso beverage from the cartridge. In addition it was observed that lack of compression of the powder in the cartridge also results in lack of flow of an espresso beverage from the cartridge. Therefore, an optimal level of compression of the powder in the cartridge should be determined in order to achieve proper flow of an espresso beverage from the cartridge. As shown here, the level of compression of the powder in the cartridge correlates inter alia with the size of the powder particles.

It is well known in the art that an addition of collagen powder to a beverage or food product renders the beverage or food product not tasty to the consumer. In this regard, the inventor has surprisingly discovered that an espresso beverage, comprising collagen peptides, according to the present invention, is much tastier, and has a much better mouth fill, than an espresso beverage not comprising collagen peptides. Not only that the present invention neutralized the unpleasant taste of an espresso beverage comprising collagen peptides, but even improved the taste and mouth fill of the espresso beverage.

Furthermore, the present invention provides a convenient delivery method of collagen peptides to a user, by preparing and ingesting an espresso beverage containing collagen peptides, while eliminating the burden of mixing a collagen powder with a coffee beverage serving, eliminating the unpleasant taste of powdered collagen peptides, and enriching the taste and mouth fill of the espresso beverage.

According to one embodiment, a cartridge containing a substance for the preparation of an espresso beverage and collagen powder may further comprise at least one additive. Examples of an additive include, but not limited to, nutritional supplement, flavor enhancer, health enhancer, and any combination thereof. Specific example of an additive include, but not limited to, lemon lime, Muscat nut, cinnamon, coffee berry, coffee berry extract, hyaluronic acid, glycyrrhizinate (licorice extract), derivative products of glycyrrhizinate, and any combination thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-42. (canceled)

43. An espresso cartridge for the preparation of an espresso beverage containing collagen peptides, the espresso cartridge comprising a portion of a substance for the preparation of an espresso beverage and a portion of collagen powder.

44. The espresso cartridge of claim 43, wherein the portion of the substance for the preparation of an espresso beverage and the portion of collagen powder are mixed.

45. The espresso cartridge of claim 43, wherein the portion of substance for the preparation of an espresso beverage and the portion of collagen powder are separated in a plurality of layers.

46. The espresso cartridge of claim 45, wherein at least one layer comprises a portion of a substance for the preparation of an espresso beverage, and at least one layer comprises a portion of collagen powder.

47. The espresso cartridge of claim 45, wherein a layer comprising a portion of a substance for the preparation of an espresso beverage is placed adjacent to an exit side of the espresso cartridge, and a layer comprising a portion of collagen powder is placed adjacent to an entry side of the espresso cartridge.

48. The espresso cartridge of claim 45, wherein a layer comprising a portion of collagen powder is placed adjacent to an exit side of the espresso cartridge, and a layer comprising a portion of a substance for the preparation of an espresso beverage is placed adjacent to an entry side of the espresso cartridge.

49. The espresso cartridge of claim 43, wherein an amount of collagen powder in the espresso cartridge is at least substantially 0.2 gr.

50. The espresso cartridge of claim 43, wherein an amount of collagen powder in the espresso cartridge is in the range of substantially 3-100% of a total amount of substance in the espresso cartridge.

51. The espresso cartridge of claim 43, wherein an amount of collagen powder in the espresso cartridge is substantially 25% of a total amount of powder in the espresso cartridge.

52. The espresso cartridge of claim 43, wherein the espresso cartridge further comprises at least one additive.

53. A method for preparing an espresso cartridge for the preparation of an espresso beverage containing collagen peptides, the method comprising inserting into an espresso cartridge a portion of a substance for the preparation of an espresso beverage and a portion of collagen powder.

54. The method of claim 53, wherein the portion of the substance for the preparation of an espresso beverage and the portion of collagen powder are mixed.

55. The method of claim 53, wherein the portion of substance for the preparation of an espresso beverage and the portion of collagen powder are separated in a plurality of layers.

56. The method of claim 55, wherein at least one layer comprises a portion of a substance for the preparation of an espresso beverage, and at least one layer comprises a portion of collagen powder.

57. The method of claim 55, wherein a layer comprising a portion of a substance for the preparation of an espresso beverage is placed adjacent to an exit side of the espresso cartridge, and a layer comprising a portion of collagen powder is placed adjacent to an entry side of the espresso cartridge.

58. The method of claim 55, wherein a layer comprising a portion of collagen powder is placed adjacent to an exit side of the espresso cartridge, and a layer comprising a portion of a substance for the preparation of an espresso beverage is placed adjacent to an entry side of the espresso cartridge.

59. The method of claim 53, wherein an amount of collagen powder in the espresso cartridge is at least substantially 0.2 gr.

60. The method of claim 53, wherein an amount of collagen powder in the espresso cartridge is in the range of substantially 3-100% of a total amount of substance in the espresso cartridge.

61. The method of claim 53, wherein an amount of collagen powder in the espresso cartridge is substantially 25% of a total amount of substance in the espresso cartridge.

62. The method of claim 53, wherein the method further comprises inserting into the espresso cartridge at least one additive.
